(19) United States
(12) Patent Application Publication
(10) Pub. No.: US 2021/0206704 A1
(43) Pub. Date: Jul. 8, 2021

(54) OXYGENATE REMOVAL FOR PARA-XYLENE PURIFICATION VIA ADSORPTION SEPARATION

(71) Applicant: UOP LLC, Des Plaines, IL (US)

(72) Inventors: Gregory B. Kuzmanich, Arlington Heights, IL (US); Nirlipt Mahapatra, Haryana (IN); Stephen W. Sohn, Arlington Heights, IL (US); Susan A. Somers, Elmhurst, IL (US); Shruti Gupta, Haryana (IN); Priyesh Jayendrakumar Jani, Haryana (IN); Pijus Kanti Roy, New Delhi (IN)

(21) Appl. No.: 16/735,302

(22) Filed: Jan. 6, 2020

(51) Int. Cl. C07C 2/86; C07C 7/163; C07C 7/00; C07C 7/12; C07C 7/09; B01J 19/24; B01D 15/18; B01D 3/14

(52) U.S. Cl. CPC C07C 2/864; C07C 7/163; C07C 7/005; C07C 7/12; B01J 2219/0004; B01J 19/245; B01D 15/1871; B01D 3/143; B01D 3/141; C07C 7/09

(57) ABSTRACT

Apparatuses and processes for producing a para-xylene stream in an aromatics complex which include a toluene methylation unit and an adsorptive separation unit. A hydrogenation zone and an oxygenate removal zone are utilized to remove oxygenates from the effluent of the toluene methylation unit. The hydrogenation zone may be a liquid phase hydrogenation zone.

### FIELD

This present disclosure relates to processes and apparatuses to aromatics complexes which produce para-xylene by toluene methylation. More specifically, the present disclosure relates to processes and apparatuses for toluene methylation in such an aromatic complex and reducing the oxygenates in the effluent from the toluene methylation.

### BACKGROUND

The xylene isomers are produced in large volumes from petroleum as feedstocks for a variety of important industrial chemicals. Currently, para-xylene, a principal feedstock for polyester production, continues to enjoy a high growth rate from a large base demand. Ortho-xylene is used to produce phthalic anhydride, which supplies high-volume but relatively mature markets. Meta-xylene is used in lesser but growing volumes for such products as plasticizers, azo dyes, and wood preservers. Ethylbenzene generally is present in xylene mixtures and is occasionally recovered for styrene production but is usually considered a less-desirable component of C8 aromatics.

Xylenes are produced from petroleum by reforming naphtha but not in sufficient volume to meet demand, thus conversion of other hydrocarbons to xylenes is necessary to increase the yield of xylenes from the feedstock. Traditional aromatics complex flow schemes are disclosed by Meyers in the HANDBOOK OF PETROLEUM REFINING PROCESSES, 2d. Edition in 1997 by McGraw-Hill.

In conventional aromatics complexes, toluene is often de-alkylated to produce benzene or selectively disproportionated to yield benzene and C8 aromatics from which the individual xylene isomers are recovered. Traditional aromatics complexes send toluene to a transalkylation zone to generate desirable xylene isomers via transalkylation of the toluene with A9+ components. A9+ components are present in both the reformate bottoms and the transalkylation effluent.

Additionally, traditional aromatics complexes may react toluene and methanol in a toluene methylation zone to produce additional xylenes. The effluent from the toluene methylation zone is generally recognized to include oxygenates and other compounds that are detrimental to existing catalysts and adsorbents of an aromatics complex. For example, U.S. Pat. No. 9,295,962 discloses a process in which the oxygenates produced in toluene methylation unit are removed by caustic washing and fractionation. This reference only discloses a method to remove acidic oxygenates with an acid dissociation constant less than 15.5. Additionally, this reference discloses caustic treatment as an adequate removal for phenolic oxygenates with acid dissociation constants of approximately 8-11. However, not as well understood is that toluene methylation produces approximate 0-50 ppm of oxygenate materials with boiling points between 80 and 192° C. that cannot be removed by caustic treatment or fractionation. These residual oxygenates have been shown to negatively impact the catalysts and adsorbents in the aromatics complex. Therefore, it is important to remove the trace oxygenates to reduce the risk of contaminating downstream units.

Current solutions are provided to remove the oxygenates from the portion of the toluene methylation effluent that are routed to the adsorbent separation zones; however, these current solutions often operate in a manner that reduces the amount of xylenes recovered. In other words, the removal of these oxygenates is at the cost of the desired products being recovered.

Therefore, it would be desirable to provide processes that provide for the effective and efficient removal of these contaminants, particularly in an aromatics complex, without negatively impacting the recovery of the desired products.

### SUMMARY OF THE INVENTION

The present invention provides various processes and configurations for an aromatics complex that effectively and efficiently remove oxygenates, as well as olefins, from a stream containing a portion of an effluent from a toluene methylation zone. The present processes removing oxygenate materials with boiling points between 80 and 192° C. from a toluene methylation effluent stream by utilizing a combined selective hydrogenation and hydrodeoxygenation chemistry in a reactor, preferably a liquid phase reactor, followed by conversion of unconverted oxygenates into heavier species across acidic clay catalyst.

In at least one aspect, the present invention may be generally characterized as providing a process for the production of para-xylene by: reacting toluene with methanol under alkylation conditions in the presence of an alkylation catalyst to provide an effluent having greater than 24% (weight) para-xylene in a xylene fraction, oxygenates, and olefins, and wherein the effluent has a Bromine Index of more than 200; selectively removing, in a subsequent hydrogenation zone, unsaturated oxygenates and olefins from at least a portion of the effluent with a hydrogenation catalyst configured to saturate olefins and convert unsaturated oxygenates into alcohols and to provide an olefin lean effluent including para-xylene and trace oxygenates, and wherein a Bromine Index of the olefin lean effluent is less than 100; selectively removing, in an oxygenate removal zone, trace oxygenates from at least a portion of the olefin lean effluent with an acidic material including polymeric resins, clays, or mixtures thereof at a temperature between 150 to 190° C. to provide an oxygenate and olefin lean effluent; and, separating a stream of para-xylene from at least a portion of the oxygenate and olefin lean effluent by adsorptive separation.

It is contemplated that the hydrogenation zone includes a liquid phase hydrogenation reactor.

It is also contemplated that the oxygenate and olefin lean effluent, after selectively removing trace unsaturated oxygenates, has a Bromine Index of less than 10.

In at least a second aspect, the present invention may generally be characterized as providing a process for the production of para-xylene by: passing a toluene stream including toluene and a methanol stream including methanol to a toluene methylation zone having a catalyst configured to, under alkylation conditions, alkylate toluene with methanol and providing a toluene methylation effluent stream having greater than 24% (weight) para-xylene in a xylene fraction, oxygenates, and olefins and wherein the toluene methylation effluent stream has a Bromine Index of more than 200; passing at least a portion of the toluene methylation effluent stream to a hydrogenation zone including a catalyst configured to, under hydrogenation conditions, selectively saturate olefins and convert unsaturated oxygenates into alcohols and providing an olefin lean toluene methylation effluent stream including para-xylene and trace oxygenates and wherein a Bromine Index of the olefin lean toluene methylation effluent stream is less than 100; passing at least a portion of the olefin lean effluent stream to an oxygenate removal zone including an acidic material including polymeric resins, clays, or mixtures thereof configured to, under removal conditions at a temperature between 150 to 190° C., selectively remove trace oxygenates and providing an oxygenate and olefin lean toluene methylation effluent stream; and passing at least a portion of the oxygenate and olefin lean toluene methylation effluent stream to an adsorptive separation zone including an adsorbent configured to, under adsorptive separation conditions, selectively adsorb and desorb para-xylene and providing a para-xylene product stream.

It is contemplated that the toluene stream having toluene is provided from a benzene/toluene fractionation zone, and wherein the process further includes: passing the toluene methylation effluent stream to the benzene/toluene fractionation zone; and, separating at least the toluene methylation effluent stream in the benzene/toluene fractionation zone into at least the toluene stream and a bottoms stream.

It is further contemplated that the benzene/toluene fractionation zone includes at least two columns.

It is also contemplated that the benzene/toluene fractionation zone includes a divided wall column.

It is contemplated that the processing also includes passing, as the portion of the toluene methylation effluent stream, the bottoms stream from the benzene/toluene fractionation zone to the hydrogenation zone. The bottoms stream from the benzene/toluene fractionation zone may be combined with a reformate splitter bottoms stream prior to the hydrogenation zone. The process may include: passing the oxygenate and olefin lean toluene methylation effluent stream to a xylene fractionation column; and separating, in the xylene fractionation column, the oxygenate and olefin lean toluene methylation effluent stream into a xylene stream and at least one other stream, wherein the xylene stream is the portion of the oxygenate and olefin lean toluene methylation effluent stream passed to the adsorptive separation zone.

It is contemplated that the processing further includes passing: the bottoms stream from the benzene/toluene fractionation zone to a xylene fractionation column; and, separating, in the xylene fractionation column, the bottoms stream from the benzene/toluene fractionation zone into a xylene stream and at least one other stream, wherein the xylene stream is the portion of the toluene methylation effluent stream passed to the hydrogenation zone. The xylene fractionation column may also receive a reformate splitter bottoms stream.

It is further contemplated that the process includes: separating, in a reformate splitter, a reformate effluent into an overhead stream, having toluene and benzene, and a bottoms stream; and, passing the toluene methylation effluent stream to the reformate splitter.

It is further contemplated that the process includes passing, as the portion of the toluene methylation effluent stream, the bottoms stream from the reformate splitter to the hydrogenation zone.

It is still further contemplated that the process includes: passing the bottoms stream from the reformate splitter to a xylene fractionation column; and, separating, in the xylene fractionation column, the bottoms stream from the from the reformate splitter into a xylene stream and at least one other stream, wherein the xylene stream is the portion of the toluene methylation effluent stream passed to the hydrogenation zone.

It is also further contemplated that the process includes: combining the toluene methylation effluent stream with a reformate stream to form a combined effluent stream; and, passing the combined effluent stream to the hydrogenation zone as the portion of the toluene methylation effluent stream passed to the hydrogenation zone. The process may further include passing the oxygenate and olefin lean toluene methylation effluent stream from the oxygenate removal zone to a reformate splitter configured to provide at least an overhead stream including toluene and a bottoms stream including para-xylene. The process may also include: passing the bottoms stream from the reformate splitter to a xylene fractionation column; and, separating, in the xylene fractionation column, the bottoms stream from the reformate splitter into a xylene stream and at least one other stream, wherein the xylene stream is the portion of the toluene methylation effluent stream passed to the hydrogenation zone.

It is contemplated that in some aspects and embodiments, the toluene methylation effluent stream is passed directly to the hydrogenation zone without being combined with any process stream.

In at least a third aspect, the present invention may be characterized as generally providing, an aromatics complex for producing para-xylene having: a toluene methylation zone having a reactor with a catalyst, the toluene methylation zone configured to receive a toluene stream and a methanol stream and configured to provide a toluene methylation effluent stream having greater than 24% (weight) para-xylene in a xylene fraction, oxygenates, and olefins, wherein the toluene methylation effluent stream has a Bromine Index of more than 200; a hydrogenation zone having a reactor with a catalyst, the hydrogenation zone configured to receive a least a portion of the toluene methylation effluent stream and configured to provide an olefin lean toluene methylation effluent stream including para-xylene and trace unsaturated oxygenates, wherein a Bromine Index of the olefin lean toluene methylation effluent stream is less than 100; an oxygenate removal zone including a reactor with an acidic material including polymeric resins, clays, or mixtures thereof, the oxygenate removal zone configured to receive at least a portion of the olefin lean toluene methylation effluent stream and configured to provide an oxygenate and olefin lean toluene methylation effluent stream, wherein a Bromine Index of the oxygenate and olefin lean toluene methylation effluent stream is zero, or less than 1; and, an adsorptive separation zone including a reactor with an adsorbent, the adsorptive separation zone configured to receive at least a portion of the oxygenate and olefin lean toluene methylation effluent stream and configured to provide a para-xylene product stream.

Additional aspects, embodiments, and details of the invention, all of which may be combinable in any manner, are set forth in the following detailed description of the invention.

### DEFINITIONS

As used herein, the term “stream”, “feed”, “product”, “part” or “portion” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. Each of the above may also include aromatic and non-aromatic hydrocarbons.

Hydrocarbon molecules may be abbreviated C1, C2, C3, Cn where “n” represents the number of carbon atoms in the one or more hydrocarbon molecules or the abbreviation may be used as an adjective for, e.g., non-aromatics or compounds Similarly, aromatic compounds may be abbreviated A6, A7, A8, An where “n” represents the number of carbon atoms in the one or more aromatic molecules. Furthermore, a “+” or “−” may be used with an abbreviated one or more hydrocarbons notation, e.g., C3+ or C3−, which is inclusive of the abbreviated one or more hydrocarbons. As an example, the abbreviation “C3+” means one or more hydrocarbon molecules of three or more carbon atoms.

As used herein, the term “zone” or “unit” can refer to an area including one or more equipment items and/or one or more sub-zones. Equipment items can include, but are not limited to, one or more reactors or reactor vessels, separation vessels, distillation towers, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally 50%, and preferably 70%, by mole, of a compound or class of compounds in a stream.

As depicted, process flow lines in the FIGURES can be referred to interchangeably as, e.g., lines, pipes, feeds, gases, products, discharges, parts, portions, or streams.

As used herein, the term “kilopascal” may be abbreviated “kPa” and the term “megapascal” may be abbreviated “MPa”, and all pressures disclosed herein are absolute.

### DETAILED DESCRIPTION OF THE DRAWINGS

One or more exemplary embodiments of the present invention will be described below in conjunction with the following drawing figures, in which:

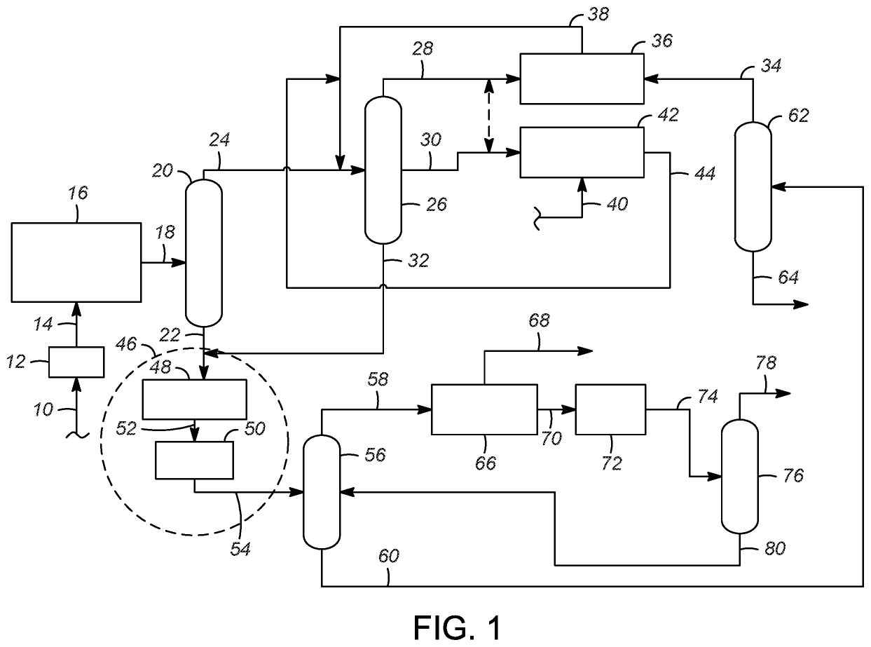

FIG. 1 shows a schematic flow diagram for an aromatics complex according one or more embodiments of the present invention;

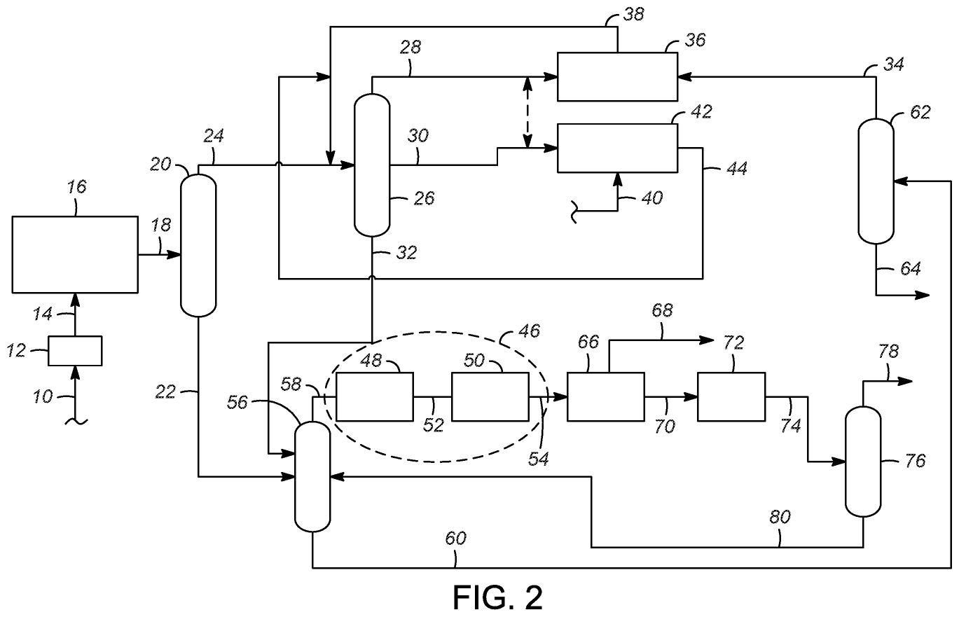

FIG. 2 shows another schematic flow diagram for an aromatics complex according one or more embodiments of the present invention;

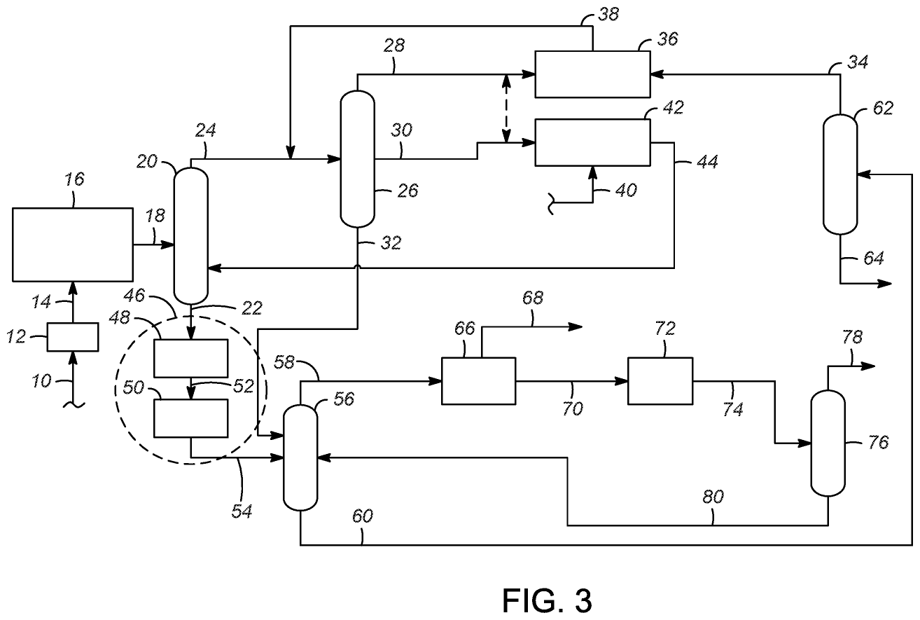

FIG. 3 shows a further schematic flow diagram for an aromatics complex according one or more embodiments of the present invention;

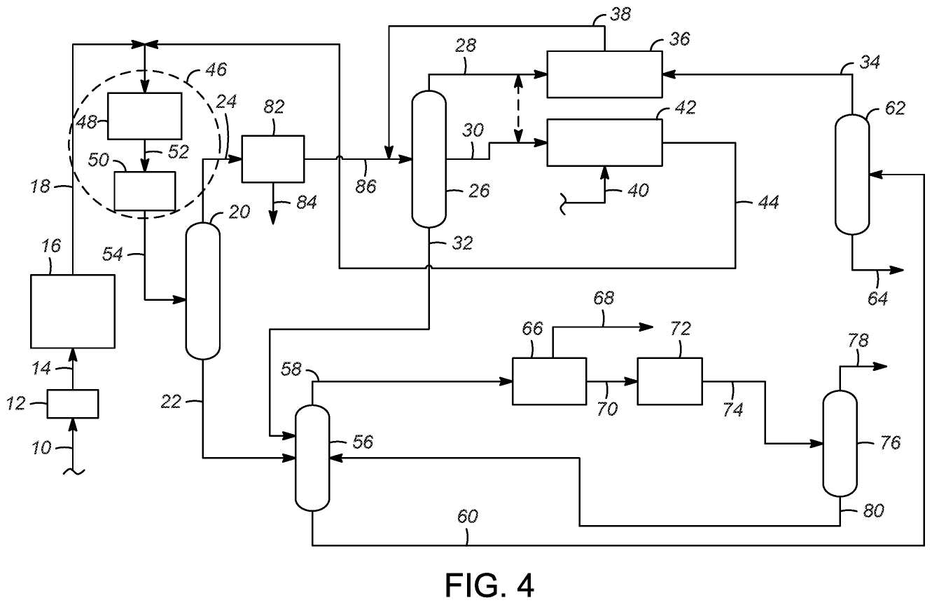

FIG. 4 shows yet another schematic flow diagram for an aromatics complex according one or more embodiments of the present invention; and,

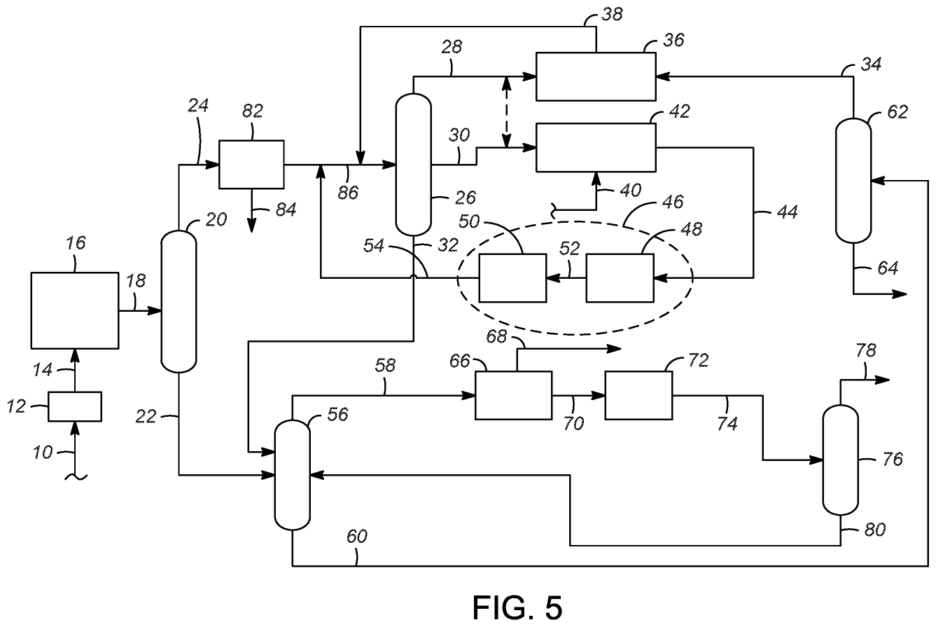

FIG. 5 shows a further schematic flow diagram for an aromatics complex according one or more embodiments of the present invention.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION

As mentioned above, the present processes and configurations for an aromatics complex utilize selective hydrogenation in a reactor, preferably a liquid phase reactor, followed by reaction of unconverted oxygenates through clay treatment. These two treatments provide for the effective and efficient removal of oxygenates, as well as olefins, from a stream containing a portion of the effluent from the toluene methylation. It is contemplated that the effluent from the toluene methylation unit combines with the reformate splitter bottoms and the combined stream is passed through a single hydrogenation reactor and then a clay treater. The combination of hydrogenation followed by clay treating ensures almost complete saturation of both olefins and oxygenates without formation of heavy aromatics and without changing the xylene compositions of aromatics stream. As an alternative, it is also contemplated that the hydrogenation and clay treating zones receive an overhead stream from a xylene fractionation column located between the adsorptive separation unit and the toluene methylation zone. It is alternately contemplated that the toluene methylation effluent is passed to the reformate splitter. The bottoms stream from the reformate splitter column contains C8+ aromatics, as well as the oxygenates, and may be passed directly, or after separation in a xylene column, to the hydrogenation and clay treating zones for treatment. Alternatively, it is further contemplated that the toluene methylation effluent and the reformate, or a C4+ portion of the reformate, are combined and then the combined effluent stream may be treated in the hydrogenation and clay treating zones to remove olefins and oxygenates. The treated stream could then be passed to the reformate splitter column. It is even further contemplated that the toluene methylation effluent could be directly treated in the hydrogenation and clay treating zones to remove olefins and oxygenates without combination with any other process stream. Once treated, the stream may be passed to the xylene column.

One of the primary benefits provided by any of the embodiments, aspects, processes and alternatives, is the removal of C5-C6 oxygenates from the toluene effluent or a portion thereof Further benefits provided by the present disclosure include an extended clay treater life, little to no aromatics yield loss, and minimal increased expenses compared to other solutions.

With these general principles in mind, one or more embodiments of the present invention will be described with the understanding that the following description is not intended to be limiting.

As shown in FIG. 1, a hydrocarbon feedstream 10 may be passed to the hydrotreating zone 12. In accordance with the instant embodiment as discussed, the hydrocarbon feedstream 10 is a naphtha stream and hence interchangeably referred to as naphtha stream. As used herein, the term “naphtha” means the hydrocarbon material boiling in the range between about 10° C. and about 200° C. atmospheric equivalent boiling point (AEBP) as determined by any standard gas chromatographic simulated distillation method such as ASTM D2887, all of which are used by the petroleum industry. The hydrocarbon material may be more contaminated and contain a greater amount of aromatic compounds than is typically found in refinery products. The typical petroleum derived naphtha contains a wide variety of different hydrocarbon types including normal paraffins, branched paraffins, olefins, naphthenes, benzene, and alkyl aromatics. Although the present embodiment is exemplified by a naphtha feedstream, the process is not limited to a naphtha feedstream, and can include any feedstream with a composition that overlaps with a naphtha feedstream.

The naphtha stream 10 may be provided to the hydrotreating zone 12 to produce a hydrotreated naphtha stream 14. As will be appreciated, the hydrotreating zone 12 may include one or more hydrotreating reactors for removing sulfur and nitrogen from the naphtha stream 10. A number of reactions take place in the hydrotreating zone 12 including hydrogenation of olefins and hydrodesulfurization of mercaptans and other organic sulfur compounds; both of which (olefins, and sulfur compounds) are present in the naphtha fractions. Examples of sulfur compounds that may be present include dimethyl sulfide, thiophenes, benzothiophenes, and the like. Further, reactions in the hydrotreating zone 12 include removal of heteroatoms, such as nitrogen and metals. Conventional hydrotreating reaction conditions are employed in the hydrotreating zone 12 which are known to one of ordinary skill in the art.

The hydrotreated naphtha stream 14 may be withdrawn from the hydrotreating zone 12 and passed to a catalytic reforming unit 16 to provide a reformate stream 18. As is known, the catalytic reforming unit 16 includes one or more reactors which receive a catalyst for promoting a reforming reaction and which typically include inter-stage heating. The reaction conditions in the catalytic reforming unit 16 may include a temperature of from about 300° C. to about 500° C., and a pressure from about 0 kPa(g) to about 3500 kPa(g).

Generally, reforming catalysts generally comprise a metal on a support. This catalyst is conventionally a dual-function catalyst that includes a metal hydrogenation-dehydrogenation catalyst on a refractory support. The support can include a porous material, such as an inorganic oxide or a molecular sieve, and a binder with a weight ratio from 1:99 to 99:1. In accordance with various embodiments, the reforming catalyst includes a noble metal including one or more of platinum, palladium, rhodium, ruthenium, osmium, and iridium. The reforming catalyst may be supported on refractory inorganic oxide support including one or more of alumina, a chlorided alumina a magnesia, a titania, a zirconia, a chromia, a zinc oxide, a thoria, a boria, a silica-alumina, a silica-magnesia, a chromia-alumina, an alumina-boria, a silica-zirconia and a zeolite.

Returning to FIG. 1, the reformate effluent 18 is passed to a reformate splitter column 20, where the components are separated by fractional distillation into, for example, a bottoms stream 22 includes C8 and heavier aromatics and an overhead stream 24 includes toluene and lighter hydrocarbons, including benzene. Although not depicted as such it is further contemplated that the reformate splitter column 20 provide an overhead steam including benzene, a sidedraw stream including toluene, and a bottoms stream including C8 and heavier aromatics.

As depicted, the overhead stream 24 is passed to a benzene/toluene fractionation zone 26 which is configured to separate the components by distillation and produce a benzene stream 28, a toluene stream 30, and A8+ stream 32 contains para-xylene, meta-xylene, ortho-xylene and ethylbenzene (discussed in more detail below). The benzene/toluene fractionation zone 26 may include a single fractionation column, a divided wall fractionation column, or use two (or more) fractionation columns to separate the components into the various streams mentioned above. As discussed below with respect to FIGS. 4 and 5, and an extractive distillation unit may be located between the reformate splitter column 20 and the benzene/toluene fractionation zone. As should be appreciated, if the reformate splitter column 20 provides an overhead stream including benzene and a sidedraw stream including toluene, the reformate splitter column 20 will comprise the benzene/toluene fractionation zone.

As shown in FIG. 1, the benzene stream 28 from the benzene/toluene fractionation zone 26, along with a heavy aromatic stream 34, may be passed to a transalkylation zone 36. The transalkylation zone 36 may include one or more reactors containing a first catalyst and being operated under transalkylation conditions. For example, the first catalyst includes at least one zeolitic component suitable for transalkylation, at least one zeolitic component suitable for dealkylation and at least one metal component suitable for hydrogenation. As is known, the transalkylation conditions may include a temperature of about 320 to about 440° C. A transalkylation effluent stream 38 having an increased amount of xylene compounds compared with the benzene stream 28 may be passed back to the benzene/toluene fractionation zone 26 to separate the components of the transalkylation effluent stream 38.

In order to further increase the yield of the para-xylene from a given reformate, the toluene stream 30 from the benzene/toluene fractionation zone 26, along with, for example, a methanol stream 40,are passed to a toluene methylation zone 42. As is known in the art, benzene and other aromatics may also be passed to the toluene methylation zone 42. Additionally, the methylation may be performed with dimethyl ether as is known.

The toluene methylation zone 42 includes a reactor having a catalyst configured to, under alkylation conditions, alkylate toluene with methanol and providing a toluene methylation effluent stream 44 having greater than the thermodynamic equilibrium 24% (weight) para-xylene in the xylene fraction, oxygenates, and olefins and wherein the toluene methylation effluent stream 44 has a Bromine Index of more than 200.

The Bromine Index (BI) is estimated with a standard UOP analytical method (UOP Method 304-90 Bromine Number and Bromine Index of Hydrocarbons by Potentiometric Titration). According to UOP Method 304-90, a “sample is dissolved in a titration solvent containing a catalyst that aids in the titration reaction. The solution is titrated potentiometrically at room temperature with either a 0.5-N (0.25-M) or 0.02-N (0.01-M) bromide-bromate solution depending upon whether bromine number or bromine index, respectively, is being determined. The titration uses a combination platinum electrode in conjunction with a recording potentiometric titrator. Bromine number or index is calculated from the volume of titrant required to reach a stable endpoint.

The toluene methylation effluent stream 44 may have a paraxylene to total xylene ratio of at least about 0.2, or preferably at least about 0.5, or more preferably about 0.8 to 0.95. Additionally, the toluene methylation effluent stream 44 may be passed back to the benzene/toluene fractionation zone 26, for example by being combined with transalkylation effluent stream 38, to separate the components of the toluene methylation effluent stream 44.

To separate para-xylene from the other xylene isomers, the A8+ stream 32 from the benzene/toluene fractionation zone 26, which includes xylenes from the reformate stream 18, as well as from the effluent streams 38, 44 from the transalkylation zone 36 and toluene methylation zone 42, may be passed, after fractionation, to a unit which includes an adsorbent for separating para-xylene. However, as discussed at the outset, oxygenates and other contaminants that may be in the A8+ stream 32 (as a result of the toluene methylation) can be detrimental to the adsorbent in such a unit. According to the various processes, a contaminant removal zone 46 that includes both a hydrogenation zone 48 and an oxygenate removal zone 50 is used to remove oxygenates and other contaminants prior to adsorbent separation.

As shown in the embodiment of FIG. 1, the A8+ stream 32, preferably along with the bottoms stream 22 from the reformate splitter column 20, may be passed to the hydrogenation zone 48. The hydrogenation zone 48 is configured to selectively remove saturated oxygenates and olefins with a hydrogenation catalyst configured to, under suitable hydrogenation conditions, saturate olefins and convert unsaturated oxygenates into alcohols. The hydrogenation zone 48 provides an olefin lean effluent stream 52 that includes xylenes, including para-xylene, and some trace oxygenates. A Bromine Index of the olefin lean effluent stream 52 may be less than 100, preferably less than 10, more preferably less than 1

The conditions of the hydrogenation zone 48 may include a temperature in the range of 50 to 200° C., a WHSV of 3 to 10 hr⁻¹, a pressure of 175 to 5,000 kPag and a hydrogen to olefins ratio between 0.5 to 4. The catalyst for the hydrogenation zone 48 includes at least one metal selected from Groups 8 to 10 of the Periodic Table on an inactive support material. Said metal is selected from Pd, Co, Ni, Ru, and mixtures thereof Said supports are selected from alumina, silica, titania, and mixtures thereof Exemplary conditions and catalysts are disclosed in U.S. Pat. No. 6,977,317.

As noted above, while the olefin lean effluent stream 52 has a lower amount or content of oxygenate compared with the A8+ stream 32, it still may contain a level that is too high for the downstream adsorbent.

Accordingly, the olefin lean effluent 52 is passed to the oxygenate removal zone 50. Although not depicted as such, one or more separation units configured to separate the components of the olefin lean effluent 52 by boiling points may be utilized. Returning to FIG. 1, the oxygenate removal zone 50 is configured to selectively remove, with an acidic material including polymeric resins, clays, or mixtures thereof under suitable conditions, trace oxygenates from at least a portion of the olefin lean effluent stream 52 to provide an oxygenate and olefin lean effluent stream 54. Clays may be selected from any suitable conditions include a temperature between 100 to 250° C., a WHSV of 0.25 to 3 hr⁻¹, and a pressure of 175 to 5,000 kPag. Acid clay material can be chosen from any attapulgus, tonsil, or montmorillonite clays. Exemplary examples include Engelhard F-24, Filtrol 24, Filtrol 25, or Filtrol 62 clays. U.S. Pat. No. 6,717,025 and U.S. Pat. Pub. No. 2004/0102670 disclose exemplary clay treatment processes for olefin removal.

The oxygenate and olefin lean effluent stream 54 has a lower level of oxygenates that is suitable for recovery of para-xylene with an adsorbent. Therefore, in the embodiment of FIG. 1, the oxygenate and olefin lean effluent stream 54 is passed to a xylene separation zone 56. The xylene separation zone 56 includes one or more fractionation columns that are configured to separate the components of the oxygenate and olefin lean effluent stream 54 stream by boiling point and provide an overhead stream 58 and a bottoms stream 60. The overhead stream 58 is a xylene stream and the bottoms stream 60 includes C9, C10, and heavier aromatics. The bottoms stream 60 may be passed to a heavy aromatic column 62 to separate the components into an overhead stream containing C9 and some of the C10 and C11 aromatics, with higher boiling compounds, primarily higher alkylaromatics, being withdrawn as a bottoms stream 64. The overhead stream from the heavy aromatic column 62 may be the heavy aromatic stream 34 discussed above that is passed to the transalkylation zone 36.

Returning to the xylene separation zone 56, the xylene stream 58 may be passed to an adsorptive separation zone 66 that includes one or more adsorbent vessels each having beds that include an adsorbent and one or more fractionation columns, typically a raffinate column and an extract column. As is known, the adsorptive separation zone 66 operates via adsorption employing a desorbent, to provide a mixture of para-xylene and desorbent to an extract column, which separates para-xylene from returned desorbent to provide a para-xylene rich stream 68. A non-equilibrium mixture of C8-aromatics raffinate and desorbent from the adsorbent vessels is sent to a raffinate column, which separates a raffinate stream 70 for isomerization from desorbent which is recycled to the adsorbent vessels.

The raffinate stream 70, a non-equilibrium mixture of xylene isomers and ethylbenzene, is passed to an isomerization zone 72 having an isomerization reactor. The isomerization reactor contains an isomerization catalyst configured to provide, under known conditions, a product approaching equilibrium concentrations of C8-aromatic isomers. An isomerization effluent stream 74 is passed to a fractionation column 76 which provides an overhead stream 78 including C7 and lighter hydrocarbons and a bottoms stream 80 including C8+ aromatics. The bottoms stream 80 is passed to the xylene separation zone 56 and separated as discussed above.

Turning to FIG. 2, another embodiment is shown in which the same units, zones, and streams are represented by the same reference numerals. In FIG. 2, the overhead stream 58 from the xylene separation zone 56, or xylene stream, is passed to the hydrogenation zone 48. The olefin lean effluent 52 is again passed to the oxygenate removal zone 50. The oxygenate and olefin lean effluent stream 54 from the oxygenate removal zone 50 is passed to the adsorptive separation zone 66. The remaining portions of this embodiment are the same as discussed above.

Turning to FIG. 3, another embodiment is shown in which the same units, zones, and streams are represented by the same reference numerals. In FIG. 3, the toluene methylation effluent stream 44 is passed to the reformate splitter column 20. Accordingly, the xylene compounds, and oxygenates and olefins from the toluene methylation zone 42 are contained in the bottoms stream 22 from the reformate splitter column 20.

Thus, the bottoms stream 22 from the reformate splitter column 20 may be passed to the hydrogenation zone 48. The olefin lean effluent 52 is again passed to the oxygenate removal zone 50. The oxygenate and olefin lean effluent stream 52 from the oxygenate removal zone 50 is passed to the xylene separation zone 56. Additionally, the A8+ stream 32 from the benzene/toluene fractionation zone 26 is passed to the xylene separation zone 56. The remaining portions of this embodiment are the same as discussed above.

In further modification of the process in FIG. 3, the contaminant removal zone 46 may be positioned downstream of the xylene separation zone 56 (as depicted in FIG. 2). Thus, the bottoms stream 22 from the reformate splitter column 20 may be passed to the xylene separation zone 56, and the overhead stream 58 from the xylene separation zone 56 may be passed to the hydrogenation zone 48.

In FIG. 4, a further embodiment is shown in which again, the same units, zones, and streams are represented by the same reference numerals. In this embodiment, the toluene methylation effluent stream 44 and the reformate 18 are passed to the hydrogenation zone 48. The olefin lean effluent 52 is again passed to the oxygenate removal zone 50. The oxygenate and olefin lean effluent stream 54 from the oxygenate removal zone 50 is passed to the reformate splitter column 20.

Accordingly, xylenes from the toluene methylation zone 42 are contained in the bottoms stream 22 from the reformate splitter column 20. In this embodiment, the overhead stream 24 from the reformate splitter column is passed to an extractive distillation unit 82 which separates a raffinate stream 84 including largely aliphatic raffinate. The remaining components from the overhead stream 24 are contained in an extract stream 86 which is passed to the benzene/toluene fractionation zone 26 and the process proceeds as described above. It should be appreciated that the extractive distillation unit 82 can be utilized in conjunction with the embodiments shown in FIGS. 1 to 3.

Turing to FIG. 5, another embodiment is shown in which the same units, zones, and streams are represented by the same reference numerals. In this embodiment, the toluene methylation effluent stream 44, without combination with any other process streams, is passed to the hydrogenation zone 48. The olefin lean effluent 52 is again passed to the oxygenate removal zone 50. The oxygenate and olefin lean effluent stream 54 from the oxygenate removal zone 50 is passed to the benzene/toluene fractionation zone 26.

In the various embodiments, the hydrogenation zone 48 and the oxygenate removal zone 50 are arranged to reduce and remove the oxygenates and olefins prior to the separation of para-xylene from a xylene stream which minimizes damaging the adsorbent typically utilized in such separating processes.

### EXAMPLES

Experimental examples of the principles of the present invention indicated oxygenates can be completely removed from the product stream while not impacting the aromatics retention or para-xylene to xylene ratio of the effluent.

To show the concepts of the present invention a Model Feed Blend with a composition given in Table 1 was passed over a reduced nickel impregnated alumina bead. The process conditions are also given in Table 1. As shown in Table 1, the hydrogenation zone converts 90+ percent of the oxygenate and olefinic material. All data was analyzed using standard gas chromatographic techniques.

TABLE 1

| Feed | | | |
|---|---|---|---|
| Benzene | 0.03 | wt % | |
| Toluene | 50.04 | wt % | |
| m-xylene | 8.97 | wt % | |
| o-xylene | 3.50 | wt % | |
| p-xylene | 3.67 | wt % | |
| Ethyl benzene | 32.64 | wt % | |
| Styrene | 0.51 | wt % | |
| DIB | 0.53 | wt % | |
| A9+ | 0.04 | wt % | |
| Non aromatics | 0.05 | wt % | |
| Unknown | 0.02 | wt % | |
| 3-Hexanone | 100 | ppm | |
| Hexanal | 100 | ppm | |
| Process Conditions | | | |
| WHSV | 5 | h⁻¹ | |
| Temperature | 50 | C | |
| Pressure | 2068 | KPa | |
| H2/Olefin | 1.57 | Mol/mol | |
| Effluent | | | |
| Time on Stream | 24 | 300 | h |
| Styrene Conversion | 100 | 100 | % |
| DIB Conversion | 86 | 85 | % |
| 3-Hexanone Conversion | 95 | 92 | % |
| Hexanal Conversion | 100 | 100 | % |

To show the concepts of the present invention a Model Feed Blend with a composition given in Table 2 was passed over an acidic montmorillonite clay. The process conditions are also given in Table 2. As shown in Table 2, the oxygenate removal zone zone converts 99+ percent of the oxygenate material. All data was analyzed using standard gas chromatographic techniques. Hexanone and hexanal in the effluent was below the lower detection limit of the gas chromotograph, which was experimentally determined to be 0.5 ppm.

TABLE 2

| Feed | | |
|---|---|---|
| Toluene | 0.02 | wt % |
| m-xylene | 4.29 | wt % |
| o-xylene | 2.23 | wt % |
| p-xylene | 90.15 | wt % |
| A9+ | 0.39 | wt % |
| Non aromatics | 2.85 | wt % |
| Unknown | 0.07 | wt % |
| 3-Hexanone | 50 | ppm |
| Hexanal | 50 | ppm |
| PX/X | 93.2 | % |
| Process Conditions | | |
| LHSV | 1.2 | h⁻¹ |
| Temperature | 150 | C |
| Pressure | 3447 | KPa |
| Effluent | | |
| Toluene | 0.08 | wt % |
| m-xylene | 4.29 | wt % |
| o-xylene | 2.24 | wt % |
| p-xylene | 90.00 | wt % |
| A9+ | 0.39 | wt % |
| Non aromatics | 2.96 | wt % |
| Unknown | 0.04 | wt % |
| 3-Hexanone | <0.5 | ppm |
| Hexanal | <0.5 | ppm |
| PX/X | 93.2 | % |
| Hexanone conversion | >99 | % |
| Hexanal Conversion | >99 | % |

Based on the results of the experiments, it is believed that complete removal of oxygenates (ketones, aldehydes, and alcohols) could be achieved by hydrogenation followed by oxygenate removal with clay treatment.

The advantages of the such a process include longer oxygenate removal life due to the minimal heavy aromatic formation.

It should be appreciated and understood by those of ordinary skill in the art that various other components such as valves, pumps, filters, coolers, etc. were not shown in the drawings as it is believed that the specifics of same are well within the knowledge of those of ordinary skill in the art and a description of same is not necessary for practicing or understanding the embodiments of the present invention.

Any of the above lines, conduits, units, devices, vessels, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring component, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

### Specific Embodiments

While the following is described in conjunction with specific embodiments, it will be understood that this description is intended to illustrate and not limit the scope of the preceding description and the appended claims.

A first embodiment of the invention is a process for the production of para-xylene comprising reacting toluene with methanol under alkylation conditions in the presence of an alkylation catalyst to provide an effluent comprising greater than 24% (weight) para-xylene in a xylene fraction, oxygenates, and olefins, and wherein the effluent comprises a Bromine Index of more than 200; selectively removing, in a subsequent hydrogenation zone, unsaturated oxygenates and olefins from at least a portion of the effluent with a hydrogenation catalyst configured to saturate olefins and convert unsaturated oxygenates into alcohols and to provide an olefin lean effluent comprising para-xylene and trace oxygenates, and wherein a Bromine Index of the olefin lean effluent is less than 100; selectively removing, in an oxygenate removal zone, trace oxygenates from at least a portion of the olefin lean effluent with an acidic material comprising polymeric resins, clays, or mixtures thereof at a temperature between 150 to 190° C. to provide an oxygenate and olefin lean effluent; and, separating a stream of para-xylene from at least a portion of the oxygenate and olefin lean effluent by adsorptive separation. An embodiment of the invention is one, any or all of prior embodiments in this paragraph up through the first embodiment in this paragraph, wherein the hydrogenation zone comprises a liquid phase hydrogenation reactor. An embodiment of the invention is one, any or all of prior embodiments in this paragraph up through the first embodiment in this paragraph, wherein the oxygenate and olefin lean effluent, after selectively removing trace unsaturated oxygenates, comprises a Bromine Index of less than 10.

A second embodiment of the invention is a process for the production of para-xylene comprising passing a toluene stream comprising toluene and a methanol stream comprising methanol to a toluene methylation zone having a catalyst configured to, under alkylation conditions, alkylate toluene with methanol and providing a toluene methylation effluent stream comprising greater than 24% (weight) para-xylene in a xylene fraction, oxygenates, and olefins and wherein the toluene methylation effluent stream comprises a Bromine Index of more than 200; passing at least a portion of the toluene methylation effluent stream to a hydrogenation zone comprising a catalyst configured to, under hydrogenation conditions, selectively saturate olefins and convert unsaturated oxygenates into alcohols and providing an olefin lean toluene methylation effluent stream comprising para-xylene and trace oxygenates and wherein a Bromine Index of the olefin lean toluene methylation effluent stream is less than 100; passing at least a portion of the olefin lean effluent stream to an oxygenate removal zone comprising an acidic material comprising polymeric resins, clays, or mixtures thereof configured to, under removal conditions at a temperature between 150 to 190° C., selectively remove trace oxygenates and providing an oxygenate and olefin lean toluene methylation effluent stream; passing at least a portion of the oxygenate and olefin lean toluene methylation effluent stream to an adsorptive separation zone comprising an adsorbent configured to, under adsorptive separation conditions, selectively adsorb and desorb para-xylene and providing a para-xylene product stream. An embodiment of the invention is one, any or all of prior embodiments in this paragraph up through the second embodiment in this paragraph wherein the toluene stream comprising toluene is provided from a benzene/toluene fractionation zone, and wherein the process further comprises passing the toluene methylation effluent stream to the benzene/toluene fractionation zone; and, separating at least the toluene methylation effluent stream in the benzene/toluene fractionation zone into at least the toluene stream and a bottoms stream. An embodiment of the invention is one, any or all of prior embodiments in this paragraph up through the second embodiment in this paragraph, wherein the benzene/toluene fractionation zone comprises at least two columns. An embodiment of the invention is one, any or all of prior embodiments in this paragraph up through the second embodiment in this paragraph, wherein the benzene/toluene fractionation zone comprises a divided wall column. An embodiment of the invention is one, any or all of prior embodiments in this paragraph up through the second embodiment in this paragraph further comprising. passing, as the portion of the toluene methylation effluent stream, the bottoms stream from the benzene/toluene fractionation zone to the hydrogenation zone. An embodiment of the invention is one, any or all of prior embodiments in this paragraph up through the second embodiment in this paragraph wherein the bottoms stream from the benzene/toluene fractionation zone is combined with a reformate splitter bottoms stream prior to the hydrogenation zone. An embodiment of the invention is one, any or all of prior embodiments in this paragraph up through the second embodiment in this paragraph further comprising passing the oxygenate and olefin lean toluene methylation effluent stream to a xylene fractionation column;

separating, in the xylene fractionation column, the oxygenate and olefin lean toluene methylation effluent stream into a xylene stream and at least one other stream, wherein the xylene stream comprises the portion of the oxygenate and olefin lean toluene methylation effluent stream passed to the adsorptive separation zone. An embodiment of the invention is one, any or all of prior embodiments in this paragraph up through the second embodiment in this paragraph further comprising. passing the bottoms stream from the benzene/toluene fractionation zone to a xylene fractionation column; and, separating, in the xylene fractionation column, the bottoms stream from the benzene/toluene fractionation zone into a xylene stream and at least one other stream, wherein the xylene stream comprises the portion of the toluene methylation effluent stream passed to the hydrogenation zone. An embodiment of the invention is one, any or all of prior embodiments in this paragraph up through the second embodiment in this paragraph, wherein the xylene fractionation column also receives a reformate splitter bottoms stream. An embodiment of the invention is one, any or all of prior embodiments in this paragraph up through the second embodiment in this paragraph further comprising separating, in a reformate splitter, a reformate effluent into an overhead stream comprising toluene and benzene and a bottoms stream; and, passing the toluene methylation effluent stream to the reformate splitter. An embodiment of the invention is one, any or all of prior embodiments in this paragraph up through the second embodiment in this paragraph further comprising passing, as the portion of the toluene methylation effluent stream, the bottoms stream from the reformate splitter to the hydrogenation zone. An embodiment of the invention is one, any or all of prior embodiments in this paragraph up through the second embodiment in this paragraph further comprising passing the bottoms stream from the reformate splitter to a xylene fractionation column; and, separating, in the xylene fractionation column, the bottoms stream from the from the reformate splitter into a xylene stream and at least one other stream, wherein the xylene stream comprises the portion of the toluene methylation effluent stream passed to the hydrogenation zone. An embodiment of the invention is one, any or all of prior embodiments in this paragraph up through the second embodiment in this paragraph further comprising combining the toluene methylation effluent stream with a reformate stream to form a combined effluent stream; and, passing the combined effluent stream to the hydrogenation zone as the portion of the toluene methylation effluent stream passed to the hydrogenation zone. An embodiment of the invention is one, any or all of prior embodiments in this paragraph up through the second embodiment in this paragraph further comprising passing the oxygenate and olefin lean toluene methylation effluent stream from the oxygenate removal zone to a reformate splitter configured to provide at least an overhead stream comprising toluene and a bottoms stream comprising para-xylene. An embodiment of the invention is one, any or all of prior embodiments in this paragraph up through the second embodiment in this paragraph further comprising passing the bottoms stream from the reformate splitter to a xylene fractionation column; and, separating, in the xylene fractionation column, the bottoms stream from the reformate splitter into a xylene stream and at least one other stream, wherein the xylene stream comprises the portion of the toluene methylation effluent stream passed to the hydrogenation zone. An embodiment of the invention is one, any or all of prior embodiments in this paragraph up through the second embodiment in this paragraph, wherein the toluene methylation effluent stream is passed directly to the hydrogenation zone without being combined with any process stream.

A second embodiment of the invention is an aromatics complex for producing para-xylene comprising a toluene methylation zone having a reactor with a catalyst, the toluene methylation zone configured to receive a toluene stream and a methanol stream and configured to provide a toluene methylation effluent stream comprising greater than 24% (weight) para-xylene in a xylene fraction, oxygenates, and olefins, wherein the toluene methylation effluent stream comprises a Bromine Index of more than 200; a hydrogenation zone having a reactor with a catalyst, the hydrogenation zone configured to receive a least a portion of the toluene methylation effluent stream and configured to provide an olefin lean toluene methylation effluent stream comprising para-xylene and trace unsaturated oxygenates, wherein a Bromine Index of the olefin lean toluene methylation effluent stream is less than 100; an oxygenate removal zone comprising a reactor with an acidic material comprising polymeric resins, clays, or mixtures thereof, the oxygenate removal zone configured to receive at least a portion of the olefin lean toluene methylation effluent stream and configured to provide an oxygenate and olefin lean toluene methylation effluent stream, wherein a Bromine Index of the oxygenate and olefin lean toluene methylation effluent stream is 0 or less than 1; and, an adsorptive separation zone comprising a reactor with an adsorbent, the adsorptive separation zone configured to receive at least a portion of the oxygenate and olefin lean toluene methylation effluent stream and configured to provide a para-xylene product stream.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. A process for the production of para-xylene comprising:
reacting toluene with methanol under alkylation conditions in the presence of an alkylation catalyst to provide an effluent comprising greater than 24% (by weight) para-xylene in a xylene fraction, oxygenates, and olefins, and wherein the effluent comprises a Bromine Index of more than 200;
selectively removing, in a subsequent hydrogenation zone, unsaturated oxygenates and olefins from at least a portion of the effluent with a hydrogenation catalyst configured to saturate olefins,. and convert unsaturated oxygenates into alcohols,. and to provide an olefin lean effluent comprising para-xylene and trace oxygenates, and wherein a Bromine Index of the olefin lean effluent is less than 100;
selectively removing, in an oxygenate removal zone, trace oxygenates from at least a portion of the olefin lean effluent with an acidic material comprising polymeric resins, clays, or mixtures thereof at a temperature between 150 to 190° C. to provide an oxygenate and olefin lean effluent; and, separating a stream of para-xylene from at least a portion of the oxygenate and olefin lean effluent by adsorptive separation.

2. The process of claim 1, wherein the hydrogenation zone comprises a liquid phase hydrogenation reactor.

3. The process of claim 1, wherein the oxygenate and olefin lean effluent, after selectively removing trace unsaturated oxygenates, comprises a Bromine Index of less than 10.

4. A process for the production of para-xylene comprising:
passing a toluene stream comprising toluene and a methanol stream comprising methanol to a toluene methylation zone having a catalyst configured to, under alkylation conditions, alkylate toluene with methanol and providing a toluene methylation effluent stream comprising greater than 24% by weight para-xylene in a xylene fraction, oxygenates, and olefins and wherein the toluene methylation effluent stream comprises a Bromine Index of more than 200;
passing at least a portion of the toluene methylation effluent stream to a hydrogenation zone comprising a catalyst configured to, under hydrogenation conditions, selectively saturate olefins, and convert unsaturated oxygenates into alcohols,. and provide an olefin lean toluene methylation effluent stream comprising para-xylene and trace oxygenates and wherein a Bromine Index of the olefin lean toluene methylation effluent stream is less than 100;
passing at least a portion of the olefin lean effluent stream to an oxygenate removal zone comprising an acidic material comprising polymeric resins, clays, or mixtures thereof configured to, under removal conditions at a temperature between 150 to 190° C., selectively remove trace oxygenates and providing an oxygenate and olefin lean toluene methylation effluent stream;
passing at least a portion of the oxygenate and olefin lean toluene methylation effluent stream to an adsorptive separation zone comprising an adsorbent configured to, under adsorptive separation conditions, selectively adsorb and desorb para-xylene and providing a para-xylene product stream.

5. The process of claim 4 wherein the toluene stream comprising toluene is provided from a benzene/toluene fractionation zone, and wherein the process further comprises:
passing the toluene methylation effluent stream to the benzene/toluene fractionation zone; and,
separating at least the toluene methylation effluent stream in the benzene/toluene fractionation zone into at least the toluene stream and a bottoms stream.

6. The process of claim 5, wherein the benzene/toluene fractionation zone comprises at least two columns.

7. The process of claim 5, wherein the benzene/toluene fractionation zone comprises a divided wall column.

8. The process of claim 5 further comprising. passing, as the portion of the toluene methylation effluent stream, the bottoms stream from the benzene/toluene fractionation zone to the hydrogenation zone.

9. The process of claim 8 wherein the bottoms stream from the benzene/toluene fractionation zone is combined with a reformate splitter bottoms stream prior to the hydrogenation zone.

10. The process of claim 8 further comprising:
passing the oxygenate and olefin lean toluene methylation effluent stream to a xylene fractionation column;
separating, in the xylene fractionation column, the oxygenate and olefin lean toluene methylation effluent stream into a xylene stream and at least one other stream, wherein the xylene stream comprises the portion of the oxygenate and olefin lean toluene methylation effluent stream passed to the adsorptive separation zone.

11. The process of claim 5 further comprising.
passing the bottoms stream from the benzene/toluene fractionation zone to a xylene fractionation column; and,
separating, in the xylene fractionation column, the bottoms stream from the benzene/toluene fractionation zone into a xylene stream and at least one other stream, wherein the xylene stream comprises the portion of the toluene methylation effluent stream passed to the hydrogenation zone.

12. The process of claim 11, wherein the xylene fractionation column also receives a reformate splitter bottoms stream.

13. The process of claim 4 further comprising:
separating, in a reformate splitter, a reformate effluent into an overhead stream comprising toluene and benzene and a bottoms stream; and, passing the toluene methylation effluent stream to the reformate splitter.

14. The process of claim 13 further comprising:
passing, as the portion of the toluene methylation effluent stream, the bottoms stream from the reformate splitter to the hydrogenation zone.

15. The process of claim 13 further comprising:
passing the bottoms stream from the reformate splitter to a xylene fractionation column; and,
separating, in the xylene fractionation column, the bottoms stream from the from the reformate splitter into a xylene stream and at least one other stream, wherein the xylene stream comprises the portion of the toluene methylation effluent stream passed to the hydrogenation zone.

16. The process of claim 6 further comprising:
combining the toluene methylation effluent stream with a reformate stream to form a combined effluent stream; and,
passing the combined effluent stream to the hydrogenation zone as the portion of the toluene methylation effluent stream passed to the hydrogenation zone.

17. The process of claim 16 further comprising:
passing the oxygenate and olefin lean toluene methylation effluent stream from the oxygenate removal zone to a reformate splitter configured to provide at least an overhead stream comprising toluene and a bottoms stream comprising para-xylene.

18. The process of claim 17 further comprising:
passing the bottoms stream from the reformate splitter to a xylene fractionation column; and,
separating, in the xylene fractionation column, the bottoms stream from the reformate splitter into a xylene stream and at least one other stream,
wherein the xylene stream comprises the portion of the toluene methylation effluent stream passed to the hydrogenation zone.

19. The process of claim 4, wherein the toluene methylation effluent stream is passed directly to the hydrogenation zone without being combined with any process stream.

20. An aromatics complex for producing para-xylene comprising:
a toluene methylation zone having a reactor with a catalyst, the toluene methylation zone configured to receive a toluene stream and a methanol stream and configured to provide a toluene methylation effluent stream comprising greater than 24% (weight) para-xylene in a xylene fraction, oxygenates, and olefins, wherein the toluene methylation effluent stream comprises a Bromine Index of more than 200;
a hydrogenation zone having a reactor with a catalyst, the hydrogenation zone configured to receive a least a portion of the toluene methylation effluent stream and configured to provide an olefin lean toluene methylation effluent stream comprising para-xylene and trace unsaturated oxygenates, wherein a Bromine Index of the olefin lean toluene methylation effluent stream is less than 100;
an oxygenate removal zone comprising a reactor with an acidic material comprising polymeric resins, clays, or mixtures thereof, the oxygenate removal zone configured to receive at least a portion of the olefin lean toluene methylation effluent stream and configured to provide an oxygenate and olefin lean toluene methylation effluent stream, wherein a Bromine Index of the oxygenate and olefin lean toluene methylation effluent stream is between 0 and 1; and,
an adsorptive separation zone comprising a reactor with an adsorbent, the adsorptive separation zone configured to receive at least a portion of the oxygenate and olefin lean toluene methylation effluent stream and configured to provide a para-xylene product stream.
